(19) United States
(12) Patent Application Publication
(10) Pub. No.: US 2007/0191908 A1
(43) Pub. Date: Aug. 16, 2007

(54) METHOD AND APPARATUS FOR STIMULATING A DENERVATED MUSCLE

(76) Inventors: Doreen K. Jacob, Pittsburgh, PA (US); Susan Tonya Stepko, Gibsonia, PA (US); Steven A. Hackworth, Pittsburgh, PA (US); Michael R. Lovell, Wexford, PA (US); Marlin H. Mickle, Pittsburgh, PA (US); Michael B. Gorin, Los Angeles, CA (US)

Correspondence Address: ECKERT SEAMANS CHERIN & MELLOTT, 600 GRANT STREET, 44TH FLOOR, PITTSBURGH, PA, 15219, US

(21) Appl. No.: 11/675,364

(22) Filed: Feb. 15, 2007

Related U.S. Application Data
(60) Provisional application No. 60/773,943, filed on Feb. 16, 2006.

(51) Int. Cl. A61N 1/18

(52) U.S. Cl. 607/48

(57) ABSTRACT

A method of stimulating a subject having a denervated muscle and a corresponding functional muscle that are responsible for producing actions, such as blinking, on first and second portions, respectively, of the subject's body. The method includes determining whether the functional muscle has contracted, generating a contraction signal if it is determined that it has contracted, and causing the denervated muscle to contract following the generation of the contraction signal. Also, an apparatus for stimulating such a subject including one or more sensing devices operatively associated with the functional muscle and one or more stimulating devices operatively associated with the denervated muscle. One or more of the sensing devices generates one or more first signals in response to activity indicating functional muscle contraction. The one or more stimulating devices are made to cause the denervated muscle to contract in response to the generation of the first signals.

### CROSS-REFERENCE TO RELATED APPLICATION

This application claims the benefit of U.S. Provisional Application No. 60/773,943, entitled “Method and Apparatus for Stimulating a Denervated Muscle,” which was filed on Feb. 16, 2006, the disclosure of which is incorporated herein by reference.

### FIELD OF THE INVENTION

The present invention relates to the treatment of disorders, such as Bell's palsy, wherein a subject has a denervated muscle, and in particular to a method and apparatus for stimulating the denervated muscle in response to a the contraction of a corresponding functional muscle of the subject.

### BACKGROUND OF THE INVENTION

Approximately 140,000 patients per year are affected by a deficit of the seventh cranial nerve, the nerve that provides signals for the muscles of facial expression for one side (left or right) of the face (as described elsewhere herein, those muscles are referred to as being “denervated”). About half of these are due to Bell's palsy, an idiopathic condition probably related to a herpes infection. Most of the Bell's patients will recover fairly good function within about 6-12 months. About 15%, however, will recover only partially and be left with significant weakness of blinking. The other 70,000 palsies are secondary to head trauma, tumors, surgical trauma and other causes. These latter patients are much less likely to recover, and much more likely to suffer permanent damage to the eye.

The current treatments for this disorder are crude and disfiguring, at best: sewing the eyelids together, connecting other nerves to the facial nerve, implanting gold weights into the upper eyelid, and others. None of these treatments, however, gives dynamic restoration of blink. Blinking, both the involuntary blinks which occur about 10-20 times per minute, and the voluntary blinks occurring when asked to close one's eyes, is critically important for protection of the eye. It functions to lubricate the ocular surface and sweep away foreign material and bacteria. Even lubrication maintains the integrity of the ocular surface, protecting it from bacterial invasion, and provides a smooth refractive surface for clear vision. Breakdown in any part of this system immediately places the eye at risk of pain, infection, and decreased vision.

In addition, a number of other disorders exist that involve unilateral paralysis of some sort, such as in the above-described Bell's palsy disorder, wherein a subject has a denervated muscle and a corresponding functional muscle. Such disorders include, without limitation, swallowing disorders, vocal cord paralysis, facial nerve dysfunction in the rest of the face (e.g., which prevents a normal smile and/or allows for saliva leakage from the paralyzed corner of the mouth), bladder dysfunction, and paralysis of half of the diaphragm (the largest muscle responsible for breathing).

There is thus a need for a method and apparatus for automatically stimulating denervated muscles in a subject that may be used to treat the above described disorders that does not include the drawbacks of the known treatment methods described above.

### SUMMARY OF THE INVENTION

In one embodiment, an apparatus is provided for stimulating a denervated muscle of a subject that has a functional muscle corresponding to the denervated muscle. The apparatus includes a sensing device located on or implanted within the body of the subject and operatively associated with the functional muscle. The sensing device includes at least one sensor for sensing a parameter, such as a voltage, current or movement, associated with the functional muscle and generating a sensor signal based thereon, and (ii) control circuitry for receiving the sensor signal, determining whether the functional muscle has contracted based on the sensor signal, and causing a first RF transmitter included in the sensing device to transmit a first RF signal if it is determined that the functional muscle has contracted. The apparatus further includes a control unit located separately from the sensing device (e.g., in a device worn by the subject, such as a pair of eyeglasses) that has an RF receiver, a controller and a second RF transmitter. The RF receiver receives the first RF signal and provides a signal based on the first RF signal to the controller. In response to receipt of the signal based on the first RF signal, the controller causes the second RF transmitter to transmit a second RF signal. The apparatus also includes a stimulating device located on or implanted within the body of the subject that has stimulating circuitry operatively associated with the denervated muscle. When the stimulating device receives the second RF signal, the stimulating circuitry provides a stimulus to the denervated muscle to cause the denervated muscle to contract.

In another embodiment, an apparatus is provided for stimulating a denervated muscle of a subject that has a functional muscle corresponding to the denervated muscle. The apparatus in this embodiment includes a sensing device located on or implanted within the body of the subject and operatively associated with the functional muscle. The sensing device includes (i) at least one sensor for sensing a parameter (such as a voltage, a current or movement) associated with the functional muscle and generating a sensor signal based thereon, and (ii) an RF transmitter for transmitting a first RF signal based on the sensor signal. The apparatus further includes a control unit located separately from the sensing device that has an RF receiver, a controller and a second RF transmitter. The RF receiver receives the first RF signal and provides a signal based on the first RF signal to the controller. The controller determines whether the functional muscle has contracted based on the signal based on the first RF signal and causes the second RF transmitter to transmit a second RF signal if the controller determines that the functional muscle has contracted. The apparatus also includes a stimulating device located on or implanted within the body of the subject that has stimulating circuitry operatively associated with the denervated muscle. When the stimulating device receives the second RF signal, the stimulating circuitry provides a stimulus to the denervated muscle to cause the denervated muscle to contract.

In either embodiment, multiple similar sensing devices and/or stimulating devices may be provided. In addition, a number of different powering methodologies may be employed. For example, power may be provided to the control unit, the sensing device or devices and the stimulating device or devices by a power storage device, such as a battery, provided therewith. Alternatively, the sensing device or devices and/or the stimulating device or devices may be powered by near-field inductive coupling with the control unit. As a further alternative, the stimulating device or devices may be powered by harvesting energy from the second RF signal that is transmitted to it/them and converting the harvested energy to DC. As still a further alternative, the sensing device or devices and/or the stimulating device or devices may be powered by harvesting energy from RF energy transmitted by a far-filed source, such as an AM radio station, and converting the harvested energy to DC.

In another embodiment, when the control circuitry of the sensing device of the apparatus determines that the functional muscle has contracted, it causes a signal to be transmitted by an antenna electrode through the subject's bodily tissue by volume conduction as described in U.S. Pat. No. 6,847,844, the disclosure of which is incorporated by reference herein. That signal is received by a similar antenna electrode provided in the stimulating device provided as part of the apparatus. Upon receipt of the signal, the stimulating device provides a stimulus to the denervated muscle to cause it to contract.

Also provided is a method of stimulating a subject having a denervated muscle and a corresponding functional muscle, wherein the functional muscle and the denervated muscle are responsible for producing actions on first and second portions, respectively, of the subject's body. The method includes determining whether the functional muscle has contracted, generating a contraction signal if it is determined that the functional muscle has contracted, and causing the denervated muscle to contract following the generation of the contraction signal.

In one particular embodiment, the method includes generating a first RF signal at a first location on or within the body of the subject and operatively associated with the functional muscle, wherein the first RF signal is based on a parameter measured in association with the functional muscle. In this embodiment, the determining step includes receiving the first RF signal at a second location and determining whether the first RF signal indicates that the functional muscle has contracted. The contraction signal in this embodiment is a second RF signal and the step of generating the contraction signal comprises generating the second RF signal only if it is determined that the first RF signal indicates that the functional muscle has contracted. The causing step in this embodiment includes receiving the second RF signal at a third location and causing the denervated muscle to contract in response to receipt of the second RF signal by providing a stimulus to the denervated muscle.

In another particular embodiment, the contraction signal is a first RF signal generated at a first location on or within the body of the subject and operatively associated with the functional muscle, wherein the causing step includes receiving the first RF signal at a second location, generating a second RF signal at the second location in response to receipt of the first RF signal, receiving the second RF signal at a third location and causing the denervated muscle to contract in response to receipt of the second RF signal by providing a stimulus to the denervated muscle.

A number of unilateral paralysis disorders may be treated with the apparatus and method described herein, including, without limitation, the following: a blinking disorder where the subject has a functional orbicularis muscle and a denervated orbicularis muscle caused by, for example, Bell's palsy; a swallowing disorder where the subject has a functional pharyngeal muscle and a denervated pharyngeal muscle; a disorder affecting the operation of the vocal cords of said subject where the functional muscle is responsible for controlling a first one or more of the vocal cords and the denervated muscle is responsible for controlling a second one or more of the vocal cords; a bladder control disorder where the functional muscle is responsible for controlling a first part of the a subject's bladder and the denervated muscle is responsible for controlling a second part of the bladder; a facial paralysis disorder where the functional muscle is responsible for moving a first part of the face of the subject and the denervated muscle is responsible for moving a second part of the face of the subject; a diaphragm paralysis disorder that adversely affects inspiratory and expiratory forces where the functional muscle includes a first portion of the diaphragm of the subject and the denervated muscle includes a second portion of the diaphragm of the subject; or any other disorder that affects a function that requires coordinated movement on both sides of the body (e.g., where the first action on the first side of the body is similar or identical to the second action on the other (opposite) side of the body).

It is an object of the present invention to provide a method and apparatus for automatically stimulating denervated muscles in a subject.

It is a further object of the present invention to provide a method and apparatus for automatically stimulating denervated muscles in a subject using an RF link with a corresponding or associated functional muscle.

It is still a further object of the present invention to provide a method and apparatus for automatically stimulating denervated muscles in a subject using volume conduction within the body of the subject as described in U.S. Pat. No. 6,847,844, the disclosure of which is incorporated by reference herein.

It is still a further object of the present invention to provide a method and apparatus for automatically stimulating denervated muscles in a subject using implantable sensing and stimulating devices.

It is still a further object of the present invention to provide a method and apparatus for automatically stimulating denervated muscles in a subject using implantable sensing devices that are powered by a near-field technique such as near field inductive coupling.

It is still a further object of the present invention to provide a method and an apparatus for treating subjects having unilateral paralysis.

It is still a further object of the present invention to a method and an apparatus for treating subjects having facial paralysis, including the inability to blink an eye.

It is still a further object of the present invention to a method and an apparatus for treating subjects having vocal cord paralysis.

It is still a further object of the present invention to a method and an apparatus for treating subjects having diaphragmatic paralysis.

It is still a further object of the present invention to a method and an apparatus for treating subjects having bladder dysfunction.

It is still a further object of the present invention to a method and an apparatus for treating subjects having pharyngeal muscle paralysis.

Therefore, it should now be apparent that the invention substantially achieves all the above aspects and advantages. Additional aspects and advantages of the invention will be set forth in the description that follows, and in part will be obvious from the description, or may be learned by practice of the invention. Moreover, the aspects and advantages of the invention may be realized and obtained by means of the instrumentalities and combinations particularly pointed out in the appended claims.

### BRIEF DESCRIPTION OF THE DRAWINGS

The accompanying drawings illustrate presently preferred embodiments of the invention, and together with the general description given above and the detailed description given below, serve to explain the principles of the invention. As shown throughout the drawings, like reference numerals designate like or corresponding parts.

FIG. 1 is a block diagram of an apparatus according to one particular embodiment of the invention for stimulating a subject that has a disorder wherein the subject has a denervated muscle and a corresponding functional muscle that are each responsible for producing an associated action on the subject's body;

FIG. 2 is a block diagram of an apparatus according to an alternate embodiment of the invention for stimulating a subject that has a disorder wherein the subject has a denervated muscle and a corresponding functional muscle that are each responsible for producing an associated action on the subject's body;

FIG. 3 is a block diagram of an apparatus according to a further alternate embodiment of the invention for stimulating a subject that has a disorder wherein the subject has a denervated muscle and a corresponding functional muscle that are each responsible for producing an associated action on the subject's body;

FIG. 4 is an isometric view of a pair of eyeglasses in which the apparatus of FIGS. 1, 2 or 3 may be implemented; and

FIG. 5 is a block diagram of an apparatus according to still a further alternate embodiment of the invention for stimulating a subject that has a disorder wherein the subject has a denervated muscle and a corresponding functional muscle wherein signals are transmitted by volume conduction.

### DESCRIPTION OF THE PREFERRED EMBODIMENTS

As used herein, the term “muscle” shall refer to a single muscle or portion thereof or a group of two or more muscles or portions of muscle tissue, such as a group of two or more muscles working cooperatively to cause a certain activity.

As used herein, the term “contract” or “contracted” shall refer one or a combination of the initiation of the contraction of a muscle or the actual contraction of a muscle to a particular degree, including full contraction and less than full contraction.

As used herein, the term “denervated” shall mean that a muscle is either partially or fully deprived of a nerve supply such that the ability of the muscle to contract normally is partially or fully impaired.

As used herein, the term “eyeglasses” shall include a device or instrument that includes corrective or non-corrective lenses or no lenses at all.

As used herein, the term “worn” shall mean carried on the person of an individual.

As used herein, the term “subject” shall refer to any member of the animal kingdom, including, but not limited to, human beings.

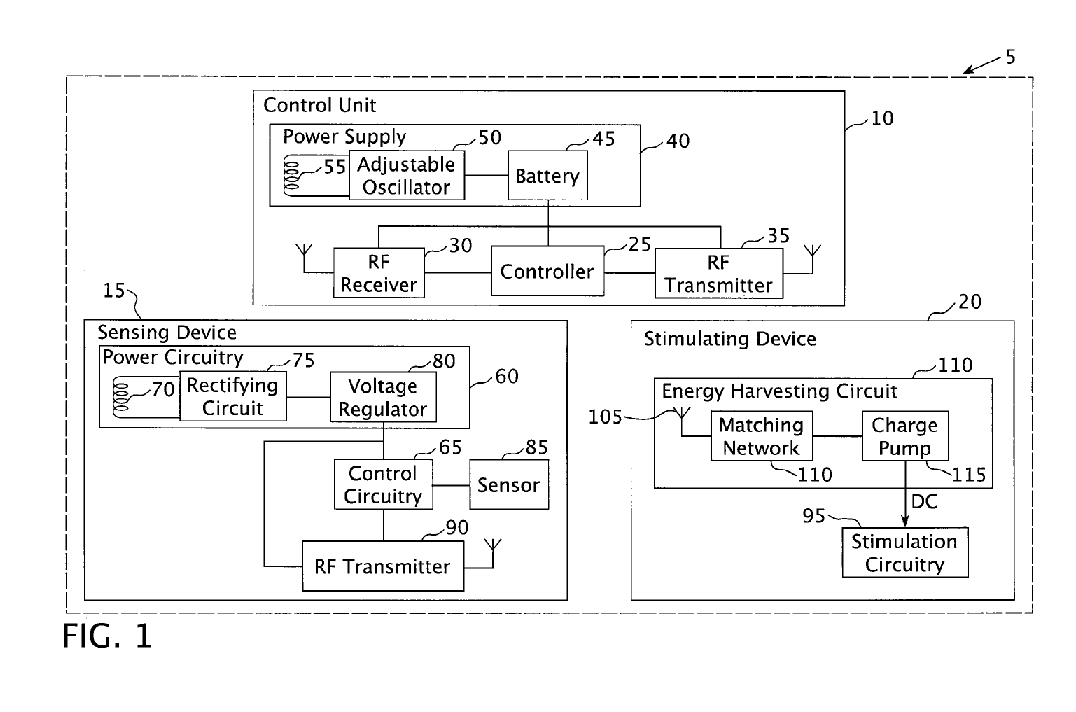

FIG. 1 is a block diagram of an apparatus 5 according to one particular embodiment of the invention for stimulating a subject that has a disorder wherein the subject has a denervated muscle and a corresponding functional muscle that are each responsible for producing an associated action on the subject's body (i.e., a first action on a first portion of the body and a second action on a second portion of the body). For example, the disorder may be Bell's palsy as described elsewhere herein, in which case the actions include blinking the subject's eyes, wherein the functional muscle is responsible causing the subject's first eye to blink and the denervated muscle is responsible for causing the subject's second eye to blink. As is known, in humans, the orbicularis muscle is the muscle that is responsible for blinking the eye, and a subject with a unilateral blinking disorder will have a functional orbicularis muscle and a denervated orbicularis muscle. Other possible unilateral paralysis disorders and associated actions that may be treated with the apparatus 5 described herein include, but are not limited to, the following: a swallowing disorder where the subject has a functional pharyngeal muscle and a denervated pharyngeal muscle; a disorder affecting the operation of the vocal cords of said subject where the functional muscle is responsible for controlling a first one or more of the vocal cords and the denervated muscle is responsible for controlling a second one or more of the vocal cords; a bladder control disorder where the functional muscle is responsible for controlling a first part of the a subject's bladder and the denervated muscle is responsible for controlling a second part of the bladder; a facial paralysis disorder where the functional muscle is responsible for moving a first part of the face of the subject and the denervated muscle is responsible for moving a second part of the face of the subject; a diaphragm paralysis disorder that adversely affects inspiratory and expiratory forces where the functional muscle includes a first portion of the diaphragm of the subject and the denervated muscle includes a second portion of the diaphragm of the subject; or any other disorder that affects a function that requires coordinated movement on both sides of the body (e.g., where the first action on the first side of the body is similar or identical to the second action on the other (opposite) side of the body).

Referring to FIG. 1, the apparatus 5 includes a control unit 10, at least one sensing device 15 located on or within (i.e., implanted) the body of the subject and operatively associated with the functional muscle in question (although only a single sensing device 15 is shown in FIG. 1 for illustrative purposes, it will be understood that multiple sensing devices 15 may be provided as part of the apparatus 5), and at least one stimulating device 20 located on or within (i.e., implanted) the body of the subject and operatively associated with the denervated muscle in question (although only a stimulating device 20 is shown in FIG. 1 for illustrative purposes, it will be understood that multiple stimulating devices 20 may be provided as part of the apparatus 5). For example, at least one sensing device 15 may be implanted within the subject's body adjacent to (and preferably in contact with) a functional orbicularis muscle or a functional pharyngeal muscle, and the least one stimulating device 20 may be implanted within the subject's body adjacent to (and preferably in contact with) a denervated orbicularis muscle or a denervated pharyngeal muscle. As described in greater detail elsewhere herein, the sensing device or devices 15 are provided to sense the contraction of the functional muscle, and the stimulating device or devices 20 are provided to cause the denervated muscle to contact when the contraction of the functional muscle is sensed. In addition, the control unit 10 is preferably included within or as part of a device worn by the subject, such as, for example, a pair of eyeglasses.

The control unit 10 includes a controller 25, which may be a microcontroller, a microprocessor, or some other type of suitable processor, including custom designed control/logic circuitry. The controller 25 is operatively coupled to an RF receiver 30 capable of receiving and preferably decoding (i.e., converting to DC logic signals) RF signals transmitted through the air, and an RF transmitter 35 capable of transmitting RF signal through the air. The RF receiver 30 and the RF transmitter 35 may be separate components, or may be combined into a single suitable RF transceiver device, many of which are known and commercially available.

The control unit 10 further includes a power supply 40 including a battery 45 for providing power to the controller 25, the RF receiver 30 and the RF transmitter 35. In addition, the battery 45 is operatively coupled to an adjustable oscillator 50 and which in turn is operatively coupled to a primary winding 55 for providing power to the sensing device 15 (or devices 15 if more than one is included) through near field inductive coupling. The definition of the near-field is generally accepted as a region that is in proximity to an antenna or another radiating structure where the electric and magnetic fields do not have a plane-wave characteristic but vary greatly from one point to another. Furthermore, the near-field can be subdivided into two regions which are named the reactive near field and the radiating near field. The reactive near-field is closest to the radiating antenna and contains almost all of the stored energy, whereas the radiating near-field is where the radiation field is dominant over the reactive field but does not possess plane-wave characteristics and is complicated in structure. This is in contrast to the far-field, which is generally defined as the region where the electromagnetic field has a plane-wave characteristic, i.e. it has a uniform distribution of the electric and magnetic field strength in planes transverse to the direction of propagation. As used herein, the terms near-field and far-field shall have the meaning provided above.

Referring to FIG. 1, in the embodiment shown therein, the near field inductive coupling is provided as follows. The adjustable oscillator 50 (a suitable example of which is the LTC6900 precision low power oscillator sold by Linear Technology Corporation of Milpitas, Calif., which is capable of generating 50% duty cycle square waves at frequencies of between 1 KHz and 20 MHz, although other types/shapes of waveforms and/or duty cycles may also be used) generates an AC signal that is provided to the primary winding 55. Furthermore, the sensing device 15 (or each sensing device 15 if appropriate) is provided with power circuitry 60 that provides a DC signal of an appropriate level for powering the control circuitry 65 provided as part of the sensing device 15 (the function of which is described in greater detail herein). As seen in FIG. 1, the power circuitry 60 includes a secondary winding 70, a voltage boosting and rectifying circuit 75 and a voltage regulator 80. In operation, when the AC signal is provided to the primary winding 55, a second AC signal is induced in the secondary winding 70 as a result of near-field inductive coupling with the primary winding 55. As will be appreciated, this requires the control unit 10 and the sensing device 15 to be located close enough to one another to allow the coupling to occur.

Furthermore, because of losses that occur in the inductive coupling, it is preferred to increase the voltage of the induced AC signal in order to provide a supply voltage of an appropriate level to the control circuitry 65. In addition, because a DC signal is employed to power the control circuitry 65, the induced AC signal is also converted to DC. Thus, the induced AC signal is provided to the voltage boosting and rectifying circuit 75, which increases the voltage of and rectifies the received AC signal. In one particular embodiment, the voltage boosting and rectifying circuit 75 is a one or more stage charge pump, sometimes referred to as a “voltage multiplier.” The DC signal that is output by the voltage boosting and rectifying circuit 75 is provided to the voltage regulator 80, which in turn provides a regulated DC voltage signal to the control circuitry 65. The voltage regulator 80 is primarily provided to resist spikes in the DC voltage signal provided to the control circuitry 65 and to resist DC voltage signals that may overdrive the control circuitry 65.

The sensor device 15 includes a sensor 85 for sensing certain activity which indicates that the functional muscle with which the sensing device 15 is associated has contracted (preferably in a manner sufficient to cause the action in question (e.g., blink) to occur). The sensor 85 is operatively coupled to the control circuitry 65 of the sensing device 15 and provides a signal thereto. The control circuitry 65 may be a processor, such as a microcontroller or microprocessor, or a custom designed logic/control circuit. Based on the signal, the control circuitry 65 makes a determination as to whether the functional muscle has contracted. As seen in FIG. 1, the sensing device 15 also includes an RF transmitter 90 that is capable of generating RF signals under the control of the control circuitry 65. Specifically, if the control circuitry 65 determines that the functional muscle has contracted, it causes the RF transmitter 90 to transmit an RF signal which, as described elsewhere herein, will ultimately result in the denervated muscle being caused to contract.

In one embodiment, the sensor 85 is a voltage sensor, such as a potential transformer or any other type of suitable known or hereafter developed voltage measuring device, that is operatively associated with (e.g., in contact with) the functional muscle and that is adapted to detect voltages that are generated in connection with the contraction of the functional muscle. The sensor 85 in this embodiment provides a detection signal to the control circuitry 65 which indicates the voltage level, if any, that is being sensed by the sensor 85. According to an aspect of this embodiment of the invention, the control circuitry 65 then determines whether the detection signal indicates that a voltage having at least a predetermined voltage level has been generated, wherein the predetermined voltage level is used as an indicator of muscle contraction. In other words, if the sensor 85 detects a voltage that is greater than some predetermined level (that is the minimum that will be considered be indicative of a contraction taking place), then the control circuitry 65 will conclude that the functional muscle has contracted and generate a signal accordingly.

In an alternative embodiment, the sensor 85 is a current sensor, such as a current transformer or any other type of suitable known or hereafter developed current measuring device, that is operatively associated with (e.g., in contact with) the functional muscle and that is adapted to detect currents that are generated in connection with the contraction of the functional muscle. Similar to the voltage sensing embodiment described above, the control circuitry 65 receives a signal from the current sensor and determines whether the signal indicates that a current having at least a predetermined level has been generated, wherein the predetermined level is used as an indicator of muscle contraction.

In still another embodiment, the sensor 85 is a motion sensor, such as an accelerometer, that is operatively associated with (e.g., in contact with) the portion of the body that is controlled by the functional muscle and that is adapted to detect movement of that body portion that is associated with the contraction of the functional muscle. The sensor 85 in this embodiment provides a detection signal to the control circuitry 65 which indicates the extent of the movement, if any, that is being sensed by the sensor 85. According to an aspect of this embodiment of the invention, the control circuitry 65 then determines whether the detection signal indicates a level of movement considered to be associated with a muscle contraction.

As seen in FIG. 1, in this particular embodiment of the apparatus 5, the stimulating device 20 includes stimulation circuitry 95 that is operatively coupled to the denervated muscle (e.g., through an electrode or some other contact that is on contact with the denervated muscle) and is structured to provide a stimulus, such as a voltage or current of an appropriate, predetermined level, to the denervated muscle to cause the denervated muscle to contract. The stimulating device 20 also includes an energy harvesting circuit 100 for providing operational power to the stimulation circuitry 95. The energy harvesting circuit 100 harvests energy that is transmitted in space. As employed herein, the term “in space” means that energy or signals are being transmitted through the air or similar medium regardless of whether the transmission is within or partially within an enclosure, as contrasted with transmission of electrical energy by a hard wired or printed circuit boards. A number of methods and apparatus for harvesting energy from space and using the harvested energy to power an electronic device are described in U.S. Pat. No. 6,289,237, entitled “Apparatus for Energizing a Remote Station and Related Method,” U.S. Pat. No. 6,615,074, entitled “Apparatus for Energizing a Remote Station and Related Method,” U.S. Pat. No. 6,856,291, entitled “Energy Harvesting Circuits and Associated Methods,” and U.S. Pat. No. 7,057,514, entitled “Antenna on a Wireless Untethered Device such as a Chip or Printed Circuit Board for Harvesting Energy from Space,” each assigned to the assignee hereof, the disclosures of which are incorporated herein by reference.

The preferred energy harvesting circuit 100 is shown in FIG. 1 and includes an antenna 105, which may be, without limitation, a square spiral antenna. The antenna 105 is electrically connected to a matching network 110, which in turn is electrically connected to a voltage boosting and rectifying circuit in the form of a charge pump 115. In operation, the antenna 105 receives energy, such as RF energy, that is transmitted in space, and provides the energy, in the form of an AC signal, to the charge pump 115 through the matching network 110. The charge pump 115 amplifies and rectifies the received AC signal to produce a DC signal. The matching network 110 preferably matches the impedance of the charge pump 115 to the impedance of the antenna 105 in a manner that optimizes the amount of energy that is harvested (i.e., maximum DC output). In one particular embodiment, the matching network 110 is an LC tank circuit formed by the inherent distributed inductance and inherent distributed capacitance of the conducing elements of the antenna 105. Such an LC tank circuit has a non-zero resistance R which results in the retransmission of some of the incident RF energy. This retransmission of energy may cause the effective area of the antenna 105 to be greater than the physical area of the antenna 105. The DC signal generated by the charge pump 115 is provided to the stimulation circuitry 95. Thus, the stimulation circuitry 95 in the stimulating device 20 in this embodiment is able to be powered without the need of an on-board power supply such as a battery. In one alternative embodiment, the DC signal generated by the charge pump 115 is used as the stimulus for causing the denervated muscle to contract, in which case the stimulation circuitry 95 may simply be an electrode or other contact for applying the Dc signal to the denervated muscle.

In operation, when the sensing device 15 determines that the functional muscle has contracted as described elsewhere herein, the RF transmitter 90, under the control of the control circuitry 65, generates and transmits a first RF signal. The first RF signal is received by the RF receiver 30 of the control unit 10, which in turn sends a signal to the controller 25 of the control unit 10. In response thereto, the controller 25 causes the RF transmitter 35 to generate and transmit a second RF signal. The second RF signal is received by the stimulating device 20, and in particular by the antenna 105 of the energy harvesting circuit 100. In response thereto, the energy harvesting circuit 100 generates a DC signal which is provided to the stimulation circuitry 95. The stimulation circuitry 95 then provides a stimulus, as described elsewhere herein, to the denervated muscle that causes the denervated muscle to contract.

According to an alternate embodiment, instead of the control circuitry 65 determining whether the parameters sensed by the sensor 85 are indicative of the contraction of the functional muscle as described above, that determination may be made by the controller 25 of the control unit 10. In particular, in this embodiment, the signals generated by the sensor 85 are converted to RF and are transmitted to the RF receiver 30 by the RF transmitter 90. The RF receiver 30 in turn provides the signal (converted back into a DC data signal) to the controller 25. Based on the received signal (i.e., the data collected by the sensor 85), the controller 25 makes a determination as to whether the functional muscle has contracted. If it is determined that the functional muscle has contracted, the controller then causes the second RF signal described above to be transmitted by the RF transmitter 35, which in turn causes the stimulus to be generated for causing the denervated muscle to contract. This embodiment may or may not omit the control circuitry 65. In addition, in this embodiment, the controller 25 may be provided with neural net software to learn the appropriate strengths of signals (e.g., voltage or current levels or extent of movement) which indicate a contraction in the functional muscle and adapt (i.e., decide when to cause the denervated muscle to contract) accordingly.

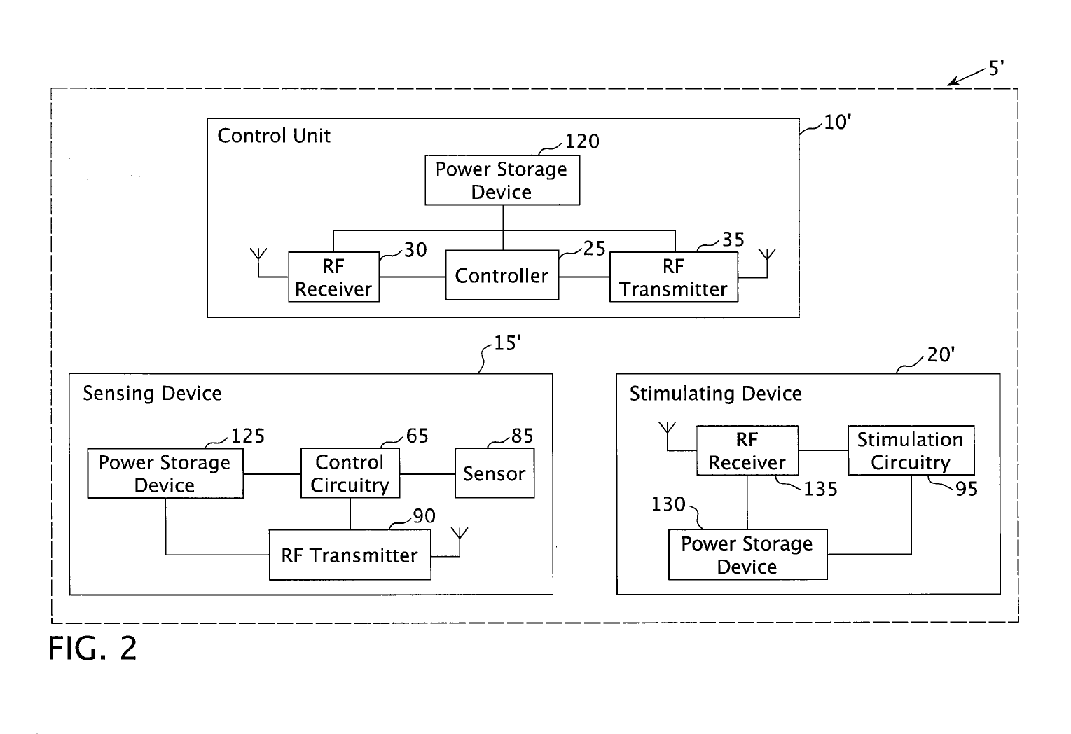

FIG. 2 is a block diagram of an apparatus 5′ according to an alternate embodiment of the invention for stimulating a subject that has a disorder wherein the subject has a denervated muscle and a corresponding functional muscle that are each responsible for producing an associated action on the subject's body. The apparatus 5′ shown in FIG. 2 is similar to the apparatus 5 shown in FIG. 1 and includes a control unit 10′, at least one sensing device 15′ located on or within (i.e., implanted) the body of the subject and operatively associated with the functional muscle in question (although only a single sensing device 15′ is shown in FIG. 2 for illustrative purposes, it will be understood that multiple sensing devices 15′ may be provided as part of the apparatus 5′), and at least one stimulating device 20′ located on or within (i.e., implanted) the body of the subject and operatively associated with the denervated muscle in question (although only a single stimulating device 20′ is shown in FIG. 2 for illustrative purposes, it will be understood that multiple stimulating devices 20′ may be provided as part of the apparatus 5′). The difference between the apparatus 5′ and the apparatus 5 is that in the apparatus 5′, power is provided to all of the components by way of power storage devices, such as batteries, that are provided with each component (instead of through near field inductive coupling and energy harvesting as in the apparatus 5). In particular, the control unit 10′ includes a power storage device 120 for powering the RF receiver 30, the controller 25, and the RF transmitter 35, the sensing device 15′ includes a power storage device 125 for powering the control circuitry 65 and the RF transmitter 90, and the stimulating device 20′ includes a power storage device 130 for powering the stimulation circuitry 95 and an RF receiver 135 that is included therein for receiving the second RF signal transmitted by the RF transmitter 35 of the control unit 10′ in the manner described elsewhere herein. Otherwise, the operation of the apparatus 5′ is identical to the operation of the apparatus 5 shown in FIG. 1.

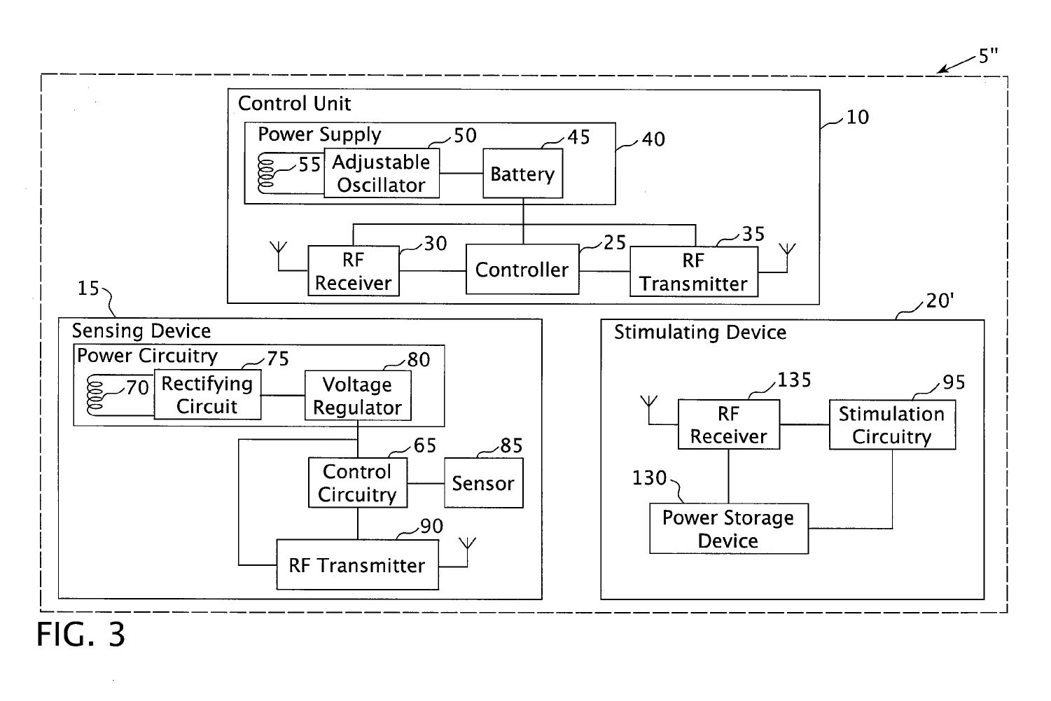

FIG. 3 is a block diagram of an apparatus 5″ according to an alternate embodiment of the invention for stimulating a subject that has a disorder wherein the subject has a denervated muscle and a corresponding functional muscle that are each responsible for producing an associated action on the subject's body. The apparatus 5″ is a hybrid of the apparatus 5 and the apparatus 5′ in which the sensing device 15 is powered by near field inductive coupling as in the apparatus 5 and the stimulating device 20′ is powered by the power storage device 130. In still further alternative embodiments, the stimulating device or devices may be powered by near field inductive coupling with the power supply 40 shown in FIG. 1 (in a manner similar to how the sensing device 15 shown in FIG. 1 is powered), or one or both of the sensing device or devices and the stimulating device or devices may be provided with an energy harvesting circuit (similar to the energy harvesting circuit 100 shown in FIG. 1) for receiving RF energy transmitted in space by a far-field source, such as, without limitation, an AM radio station, and converting the received RF energy into DC power for providing power to the sensing device or stimulating device, as the case may be.

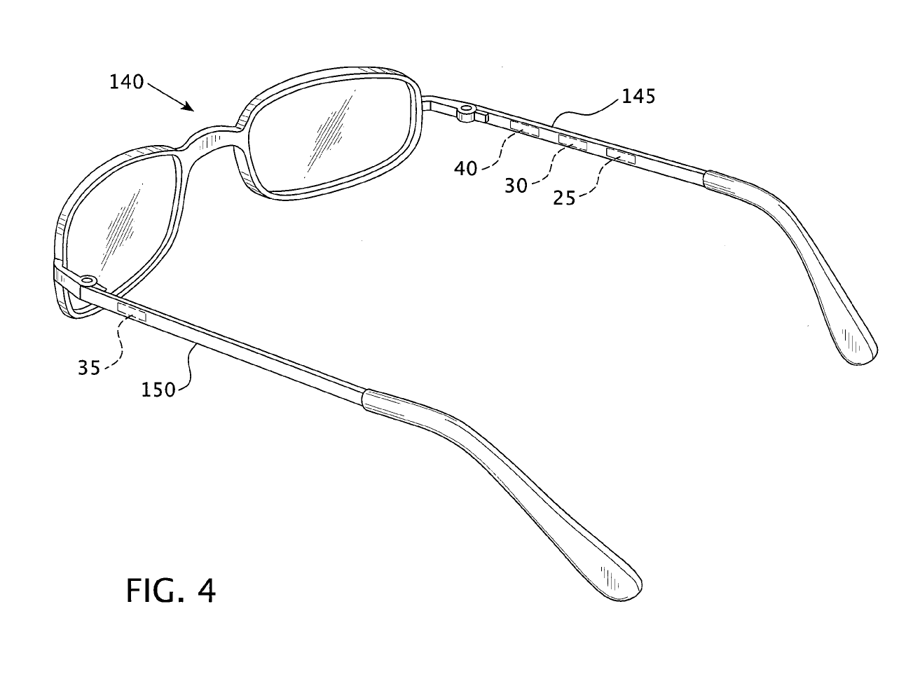

As noted elsewhere herein, the apparatus 5, 5′, or 5″ may be used to treat a blinking disorder and may be implemented in a fashion wherein the control unit 10 is formed as part of a pair of eyeglasses, such as eyeglasses 140 shown in FIG. 4, and wherein the sensing device or devices 15 (or 15′) and the stimulating device or devices 20 (or 20′) are implanted within the eyelid of the subject. In such an implementation, it is preferred to mount at least the RF receiver 30 of the control unit 10 on or within a first portion 145 of the eyeglasses 140 that is adjacent to the eye having the functional muscle (i.e., near the sensing device or devices 15 (or 15′)) and to mount at least the RF transmitter 35 of the control unit 10 on or within a second portion 150 of the eyeglasses 140 that is adjacent to the eye having the denervated muscle (i.e., near the stimulating device or devices 20 (or 20′)) to facilitate the RF transmissions described herein and, where appropriate, to facilitate the near field inductive coupling described herein. The various components of the control unit 10 may then be operatively coupled to one another as described herein by running wires or other suitable conductors (not shown) on or with the frame of the eyeglasses 140.

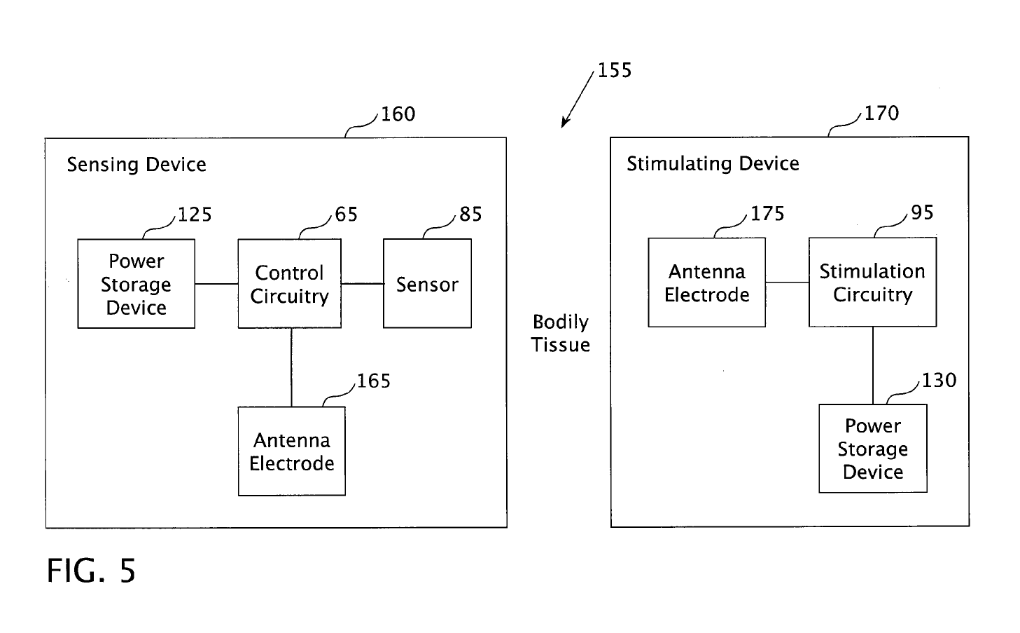

Still a further alternate embodiment of an apparatus 155 for stimulating a subject that has a disorder wherein the subject has a denervated muscle and a corresponding functional muscle is shown in FIG. 5. As seen in FIG. 5, the apparatus 155 does not include a control unit such as control unit 10 shown in FIG. 1. Instead, in this embodiment, when the control circuitry 65 of the sensing device 160 (similar to the sensing device 15′ of FIG. 2) determines that the functional muscle has contracted in the manner described elsewhere herein, it causes a signal to be transmitted by an antenna electrode 165 through the subject's bodily tissue by volume conduction as described in U.S. Pat. No. 6,847,844, the disclosure of which is incorporated by reference herein. That signal is received by a similar antenna electrode 175 provided in the stimulating device 170 (similar to the stimulating device 20′ of FIG. 2) provided as part of the apparatus 155. Upon receipt of the signal, the stimulating device 170 provides a stimulus, as described elsewhere herein, to the denervated muscle to cause it to contract. Preferably, in this embodiment, both the sensing device 160 and the stimulating device 170 are implanted, although this is not required (e.g., one or both could be located on the surface of the subject's body). Alternatively, a control unit having a power supply similar to the power supply 40 (FIG. 1) may be provided in order to power either or both of the sensing device 160 and the stimulating device 170 by near-field inductive coupling as described elsewhere herein (in which case the sensing device 160 and/or the stimulating device 170 would be provided with a power circuit similar to the power circuit 60 shown in FIG. 1).

While preferred embodiments of the invention have been described and illustrated above, it should be understood that these are exemplary of the invention and are not to be considered as limiting. Additions, deletions, substitutions, and other modifications can be made without departing from the spirit or scope of the present invention. Accordingly, the invention is not to be considered as limited by the foregoing description but is only limited by the scope of the appended claims.

What is claimed is:

1. An apparatus for stimulating a subject having a denervated muscle and a corresponding functional muscle, said functional muscle and said denervated muscle being responsible for producing actions on first and second portions, respectively, of said subject's body, comprising:
one or more sensing devices located on or within the body of said subject and operatively associated with said functional muscle, one or more of said one or more sensing devices generating one or more first signals in response to certain activity indicating that said functional muscle has contracted, wherein each of said one or more sensing devices includes a first RF transmitter, wherein the one or more first signals are each an RF signal transmitted by a respective first RF transmitter;
one or more stimulating devices located on or within the body of said subject and operatively associated with said denervated muscle; and
a control unit including: (i) a controller, (ii) a power supply, wherein power is provided to said one or more sensing devices through near field inductive coupling between said power supply and each of said sensing devices, (iii) an RF receiver for receiving said one or more first signals and providing said one or more first signals to said controller, and (iv) a second RF transmitter;
wherein when said controller receives said one or more first signals said controller causes a second signal to be generated in response thereto, said second signal being an RF signal transmitted by said second RF transmitter and received by said one or more stimulating devices, said second signal causing said one or more stimulating devices to cause said denervated muscle to contract.

2. The apparatus according to claim 1, wherein each of said one or more sensing devices includes: (i) at least one sensor for sensing a parameter associated with said functional muscle and generating a sensor signal based thereon, and (ii) control circuitry for determining whether said functional muscle has contracted based on said sensor signal, wherein each of the one or more first signals is generated only if the control circuitry of the associated one of the one or more sensing devices determines that the associated sensor signal indicates that the functional muscle has contracted.

3. The apparatus according to claim 1, wherein each of said one or more sensing devices includes at least one sensor for sensing a parameter associated with said functional muscle and generating a sensor signal based thereon, wherein each of said one or more sensing devices transmits a sensor-based RF signal based on its associated sensor signal to said control unit, wherein said transmitted sensor-based RF signals include the one or more first signals only if said functional muscle has contracted, and wherein said controller determines whether said sensor-based RF signals include the one or more first signals and generates said second signal only if it is determined that said sensor-based RF signals include the one or more first signals.

4. The apparatus according to claim 1, wherein said one or more sensing devices each include a voltage sensor, and wherein said certain activity indicating that said functional muscle has contracted comprises the production of a voltage having at least a predetermined voltage level.

5. The apparatus according to claim 1, wherein said one or more sensing devices each include a motion sensor, and wherein said certain activity indicating that said functional muscle has contracted comprises at least a predetermined amount of movement of a portion of the body of said subject that is controlled by said functional muscle.

6. The apparatus according to claim 5, wherein each said motion sensor is an accelerometer.

7. The apparatus according to claim 1, wherein said one or more sensing devices each include a current sensor, and wherein said certain activity indicating that said functional muscle has contracted comprises the production of a current having at least a predetermined current level.

8. The apparatus according to claim 1, wherein said control unit is part of a device worn by said subject.

9. The apparatus according to claim 1, wherein said actions comprise blinking a first eye of said subject and blinking a second eye of said subject, wherein said functional muscle is responsible for causing said first eye to blink and said denervated muscle is responsible for causing said second eye to blink, wherein said one or more sensing devices are attached to the eyelid of said first eye and said one or more stimulating devices are attached to the eyelid of said second eye.

10. The apparatus according to claim 9, wherein said one or more sensing devices are implanted within the eyelid of said first eye and said one or more stimulating devices are implanted within the eyelid of said second eye.

11. The apparatus according to claim 10, wherein said control unit is part of a device worn by said subject.

12. The apparatus according to claim 11, wherein said RF receiver is located on or within a first portion of said device that is adjacent to said first eye when said device is worn by said subject and wherein said second RF transmitter is located on or within a second portion of said device that is adjacent to said second eye when said device is worn by said subject.

13. The apparatus according to claim 12, wherein said device is a pair of eyeglasses.

14. The apparatus according to claim 1, wherein said actions cause said subject to swallow.

15. The apparatus according to claim 14, wherein said functional muscle comprises a first pharyngeal muscle and said denervated muscle comprises a second pharyngeal muscle.

16. The apparatus according to claim 1, wherein said actions comprise operation of the vocal cords of said subject.

17. The apparatus according to claim 16, wherein said functional muscle is responsible for controlling a first one or more of said vocal cords and said denervated muscle is responsible for controlling a second one or more of said vocal cords.

18. The apparatus according to claim 1, wherein said actions comprise control of a first part and a second part of the bladder of said subject.

19. The apparatus according to claim 18, wherein said functional muscle is responsible for controlling said first part of said bladder and said denervated muscle is responsible for controlling said second part of said bladder.

20. The apparatus according to claim 1, wherein said actions comprise moving a first part of the face of said subject and moving a second part of the face of said subject.

21. The apparatus according to claim 20, wherein said functional muscle is responsible for moving said first part of the face of said subject and said denervated muscle is responsible for moving said second part of the face of said subject.

22. The apparatus according to claim 1, wherein said actions comprise providing inspiratory and expiratory forces for said subject.

23. The apparatus according to claim 22, wherein said functional muscle comprises a first portion of the diaphragm of said subject and said denervated muscle comprises a second portion of the diaphragm of said subject.

24. The apparatus according to claim 1, wherein said one or more sensing devices and said one or more stimulating devices are implanted within the body of said subject.

25. The apparatus according to claim 1, wherein each of said one or more stimulating devices causes said denervated muscle to contract by providing a stimulus to said denervated muscle.

26. The apparatus according to claim 25, wherein each of said stimulating devices includes a circuit for providing a voltage having at least a predetermined voltage level, and wherein said stimulus comprises said voltage.

27. The apparatus according to claim 25, wherein each of said stimulating devices includes a circuit for providing a current having at least a predetermined current level, and wherein said stimulus comprises said current.

28. The apparatus according to claim 1, wherein said power supply includes an oscillator that generates an AC signal and a primary winding, wherein each of said one or more sensing devices includes a power circuit including a secondary winding, and wherein said AC signal induces a second AC signal in each said secondary winding that is converted into a DC signal by the power circuit associated with the secondary winding.

29. The apparatus according to claim 1, wherein each of said stimulating devices includes an antenna and conversion circuitry for converting said second signal into a DC signal.

30. The apparatus according to claim 29, wherein said DC signal is used to provide a stimulus to said denervated muscle, said stimulus causing said denervated muscle to contract.

31. The apparatus according to claim 30, wherein each of said stimulating devices includes stimulation circuitry for providing said stimulus and wherein said DC signal is used to power said stimulation circuitry.

32. The apparatus according to claim 29, wherein said conversion circuitry of each of said stimulating devices is a rectifier/charge pump.

33. The apparatus according to claim 1, wherein said subject is a human.

34. The apparatus according to claim 1, wherein said subject is an animal.

35. The apparatus according to claim 1, wherein said subject has a unilateral paralysis, wherein said first portion and said second portion of said subject's body are on opposite sides of said subject's body, and wherein said actions comprises similar actions.

36. The apparatus according to claim 1, wherein said denervated muscle and said functional muscle each comprises a group of two or more individual muscles.

37. The apparatus according to claim 1, wherein said denervated muscle and said functional muscle each comprises a collection of muscle tissue.

38. The apparatus according to claim 1, wherein said one or more sensing devices comprise a single sensing device and wherein said one or more first signals is a single first signal.

39. The apparatus according to claim 38, wherein said or one or more stimulating devices comprise a plurality of stimulating devices.

40. The apparatus according to claim 1, wherein said one or more sensing devices comprise a plurality of sensing devices.

41. The apparatus according to claim 40, wherein said one or more stimulating devices comprise a plurality of stimulating devices.

42. The apparatus according to claim 1, wherein power is also provided to said one or more stimulating devices through near field inductive coupling between said power supply and each of said stimulating devices.

43. The apparatus according to claim 1, wherein each of said stimulating devices includes: (i) an energy harvesting circuit including an antenna and conversion circuitry for receiving RF energy transmitted by a far-field source and converting said received RF energy into DC power, and (ii) stimulation circuitry for providing a stimulus to said denervated muscle to cause said denervated muscle to contract, said stimulation circuitry for each stimulating device being powered by the DC power generated by the stimulating device.

44. An apparatus for stimulating a subject having a denervated muscle and a corresponding functional muscle, said functional muscle and said denervated muscle being responsible for producing actions on first and second portions, respectively, of said subject's body, comprising:
one or more sensing devices located on or within the body of said subject and operatively associated with said functional muscle, one or more of said one or more sensing devices generating one or more first signals in response to certain activity indicating that said functional muscle has contracted, wherein each of said one or more sensing devices includes a first RF transmitter, wherein the one or more first signals are each an RF signal transmitted by a respective first RF transmitter;
one or more stimulating devices located on or within the body of said subject and operatively associated with said denervated muscle; and
a control unit including: (i) a controller, (ii) an RF receiver for receiving said one or more first signals and providing said one or more first signals to said controller, and (iii) a second RF transmitter;
wherein when said controller receives said one or more first signals said controller causes a second signal to be generated in response thereto, said second signal being an RF signal transmitted by said second RF transmitter, wherein each of said stimulating devices includes an antenna for receiving said second signal and conversion circuitry for converting said second signal into a DC signal, and wherein said DC signal is used to provide a stimulus to said denervated muscle that causes said denervated muscle to contract.

45. The apparatus according to claim 44, wherein each of said sensing devices includes an energy harvesting circuit including an antenna and conversion circuitry for receiving RF energy transmitted by a far-field source and converting said received RF energy into DC power for powering the sensing device.

46. The apparatus according to claim 44, wherein each of said stimulating devices includes stimulation circuitry for providing said stimulus and wherein said DC signal is used to power said stimulation circuitry.

47. The apparatus according to claim 44, wherein each of said one or more sensing devices includes: (i) at least one sensor for sensing a parameter associated with said functional muscle and generating a sensor signal based thereon, and (ii) control circuitry for determining whether said functional muscle has contracted based on said sensor signal, wherein each of the one or more first signals is generated only if the control circuitry of the associated one of the one or more sensing devices determines that the associated sensor signal indicates that the functional muscle has contracted.

48. The apparatus according to claim 44, wherein each of said one or more sensing devices includes at least one sensor for sensing a parameter associated with said functional muscle and generating a sensor signal based thereon, wherein each of said one or more sensing devices transmits a sensor-based RF signal based on its associated sensor signal to said control unit, wherein said transmitted sensor-based RF signals include the one or more first signals only if said functional muscle has contracted, and wherein said controller determines whether said sensor-based RF signals include the one or more first signals and generates said second signal only if it is determined that said sensor-based RF signals include the one or more first signals.

49. The apparatus according to claim 44, wherein said one or more sensing devices each include a voltage sensor, and wherein said certain activity indicating that said functional muscle has contracted comprises the production of a voltage having at least a predetermined voltage level.

50. The apparatus according to claim 44, wherein said one or more sensing devices each includes a motion sensor, and wherein said certain activity indicating that said functional muscle has contracted comprises at least a predetermined amount of movement of a portion of the body of said subject that is controlled by said functional muscle.

51. The apparatus according to claim 50, wherein each said motion sensor is an accelerometer.

52. The apparatus according to claim 44, wherein said one or more sensing devices each include a current sensor, and wherein said certain activity indicating that said functional muscle has contracted comprises the production of a current having at least a predetermined current level.

53. The apparatus according to claim 44, wherein said control unit is part of a device worn by said subject.

54. The apparatus according to claim 44, wherein said actions comprise blinking a first eye of said subject and blinking a second eye of said subject, wherein said functional muscle is responsible for causing said first eye to blink and said denervated muscle is responsible for causing said second eye to blink, wherein said one or more sensing devices are attached to the eyelid of said first eye and said one or more stimulating devices are attached to the eyelid of said second eye.

55. The apparatus according to claim 54, wherein said one or more sensing devices are implanted within the eyelid of said first eye and said one or more stimulating devices are implanted within the eyelid of said second eye.

56. The apparatus according to claim 55, wherein said control unit is part of a device worn by said subject.

57. The apparatus according to claim 56, wherein said RF receiver is located on or within a first portion of said device that is adjacent to said first eye when said device is worn by said subject and wherein said second RF transmitter is located on or within a second portion of said device that is adjacent to said second eye when said device is worn by said subject.

58. The apparatus according to claim 57, wherein said device is a pair of eyeglasses.

59. The apparatus according to claim 44, wherein said actions cause said subject to swallow.

60. The apparatus according to claim 59, wherein said functional muscle comprises a first pharyngeal muscle and said denervated muscle comprises a second pharyngeal muscle.

61. The apparatus according to claim 44, wherein said actions comprise operation of the vocal cords of said subject.

62. The apparatus according to claim 61, wherein said functional muscle is responsible for controlling a first one or more of said vocal cords and said denervated muscle is responsible for controlling a second one or more of said vocal cords.

63. The apparatus according to claim 44, wherein said actions comprise control of a first part and a second part of the bladder of said subject.

64. The apparatus according to claim 63, wherein said functional muscle is responsible for controlling said first part of said bladder and said denervated muscle is responsible for controlling said second part of said bladder.

65. The apparatus according to claim 44, wherein said actions comprise moving a first part of the face of said subject and moving a second part of the face of said subject.

66. The apparatus according to claim 65, wherein said functional muscle is responsible for moving said first part of the face of said subject and said denervated muscle is responsible for moving said second part of the face of said subject.

67. The apparatus according to claim 44, wherein said actions comprise providing inspiratory and expiratory forces for said subject.

68. The apparatus according to claim 67, wherein said functional muscle comprises a first portion of the diaphragm of said subject and said denervated muscle comprises a second portion of the diaphragm of said subject.

69. The apparatus according to claim 44, wherein said one or more sensing devices and said one or more stimulating devices are implanted within the body of said subject.

70. The apparatus according to claim 44, wherein each of said one or more stimulating devices causes said denervated muscle to contract by providing a stimulus to said denervated muscle.

71. The apparatus according to claim 70, wherein each of said stimulating devices include a circuit for providing a voltage having at least a predetermined voltage level, and wherein said stimulus comprises said voltage.

72. The apparatus according to claim 70, wherein each of said stimulating devices includes a circuit for providing a current having at least a predetermined current level, and wherein said stimulus comprises said current.

73. The apparatus according to claim 44, wherein said conversion circuitry of each of said stimulating devices is a rectifier/charge pump.

74. The apparatus according to claim 44, wherein said subject is a human.

75. The apparatus according to claim 44, wherein said subject is an animal.

76. The apparatus according to claim 44, wherein said subject has a unilateral paralysis, wherein said first portion and said second portion of said subject's body are on opposite sides of said subject's body, and wherein said actions comprises similar actions.

77. The apparatus according to claim 44, wherein said denervated muscle and said functional muscle each comprises a group of two or more individual muscles.

78. The apparatus according to claim 44, wherein said denervated muscle and said functional muscle each comprises a collection of muscle tissue.

79. The apparatus according to claim 44, wherein said one or more sensing devices comprise a single sensing device and wherein said one or more first signals is a single first signal.

80. The apparatus according to claim 79, wherein said one or more stimulating devices comprise a plurality of stimulating devices.

81. The apparatus according to claim 44, wherein said one or more sensing devices comprise a plurality of sensing devices.

82. The apparatus according to claim 81, wherein said one or more stimulating devices comprise a plurality of stimulating devices.

83. An apparatus for stimulating a denervated muscle of a subject, said subject having a functional muscle corresponding to said denervated muscle, comprising:
a sensing device located on or implanted within the body of said subject and operatively associated with said functional muscle, said sensing device including:
(i) at least one sensor for sensing a parameter associated with said functional muscle and generating a sensor signal based thereon, and
(ii) control circuitry for receiving said sensor signal, determining whether said functional muscle has contracted based on said sensor signal, and causing a first RF transmitter included in said sensing device to transmit a first RF signal if it is determined that said functional muscle has contracted;
a control unit located separately from said sensing device, said control unit having a power supply, wherein power is provided to said sensing device through near field inductive coupling between said power supply and said sensing device, an RF receiver, a controller and a second RF transmitter, wherein said RF receiver receives said first RF signal and provides a signal based on said first RF signal to said controller, and wherein in response to receipt of said signal based on said first RF signal said controller causes said second RF transmitter to transmit a second RF signal; and
a stimulating device located on or implanted within the body of said subject, said stimulating device having stimulation circuitry operatively associated with said denervated muscle, an antenna for receiving said second RF signal and conversion circuitry for converting said second RF signal into a DC signal provided to said stimulation circuitry for powering said stimulation circuitry, wherein when said stimulating device receives said DC signal said stimulation circuitry provides a stimulus to said denervated muscle to cause said denervated muscle to contract.

84. An apparatus for stimulating a denervated muscle of a subject, said subject having a functional muscle corresponding to said denervated muscle, comprising:
a sensing device located on or implanted within the body of said subject and operatively associated with said functional muscle, said sensing device including:
(i) at least one sensor for sensing a parameter associated with said functional muscle and generating a sensor signal based thereon, and
(ii) an RF transmitter for transmitting a first RF signal based on said sensor signal;
a control unit located separately from said sensing device, said control unit having a power supply, wherein power is provided to said sensing device through near field inductive coupling between said power supply and said sensing device, an RF receiver, a controller and a second RF transmitter, wherein said RF receiver receives said first RF signal and provides a signal based on said first RF signal to said controller, wherein said controller determines whether said functional muscle has contracted based on said signal based on said first RF signal and causes said second RF transmitter to transmit a second RF signal if said controller determines that said functional muscle has contracted; and
a stimulating device located on or implanted within the body of said subject, said stimulating device having stimulation circuitry operatively associated with said denervated muscle, an antenna for receiving said second RF signal and conversion circuitry for converting said second RF signal into a DC signal provided to said stimulation circuitry for powering said stimulation circuitry, wherein when said stimulating device receives said DC power said stimulation circuitry provides a stimulus to said denervated muscle to cause said denervated muscle to contract.

85. The apparatus according to claim 83, wherein said controller includes neural net software and employs said neural net software to adaptively determine whether said functional muscle has contracted based on said signal based on said first RF signal.

86. An apparatus for stimulating a subject having a denervated muscle and a corresponding functional muscle, said functional muscle and said denervated muscle being responsible for producing actions on first and second portions, respectively, of said subject's body, comprising:
a sensing device located on or within the body of said subject and operatively associated with said functional muscle, said sensing device generating a first signal in response to certain activity indicating that said functional muscle has contracted, said sensing device including a first antenna electrode; and
a stimulating device located on or within the body of said subject and operatively associated with said denervated muscle, said stimulating device including a second antenna electrode;
wherein said first signal is transmitted from said first antenna electrode to said second antenna electrode within the body of said subject by volume conduction, wherein said stimulating device is made to cause said denervated muscle to contract in response to receipt of said first signal by said second antenna electrode.

87. An apparatus for stimulating a denervated muscle of a subject, said subject having a functional muscle corresponding to said denervated muscle, comprising:
a sensing device located on or implanted within the body of said subject and operatively associated with said functional muscle, said sensing device including:
(i) at least one sensor for sensing a parameter associated with said functional muscle and generating a sensor signal based thereon,
(ii) control circuitry for receiving said sensor signal, determining whether said functional muscle has contracted based on said sensor signal, and causing a first RF transmitter included in said sensing device to transmit a first RF signal if it is determined that said functional muscle has contracted, and
(iii) an energy harvesting circuit including an antenna and conversion circuitry for receiving RF energy transmitted by a far-field source and converting the received RF energy into DC power for powering said control circuitry;
a control unit located separately from said sensing device, said control unit having an RF receiver, a controller and a second RF transmitter, wherein said RF receiver receives said first RF signal and provides a signal based on said first RF signal to said controller, and wherein in response to receipt of said signal based on said first RF signal said controller causes said second RF transmitter to transmit a second RF signal; and
a stimulating device located on or implanted within the body of said subject, said stimulating device having stimulation circuitry operatively associated with said denervated muscle, and an energy harvesting circuit including an antenna and conversion circuitry for receiving said RF energy and converting the received RF energy into DC power for powering said stimulation circuitry, wherein when said stimulating device receives said second RF signal said stimulation circuitry provides a stimulus to said denervated muscle to cause said denervated muscle to contract.

88. An apparatus for stimulating a denervated muscle of a subject, said subject having a functional muscle corresponding to said denervated muscle, comprising:
a sensing device located on or implanted within the body of said subject and operatively associated with said functional muscle, said sensing device including:
(i) at least one sensor for sensing a parameter associated with said functional muscle and generating a sensor signal based thereon,
(ii) an RF transmitter for transmitting a first RF signal based on said sensor signal, and
(iii) an energy harvesting circuit including an antenna and conversion circuitry for receiving RF energy transmitted by a far-field source and converting the received RF energy into DC power for powering said RF transmitter;
a control unit located separately from said sensing device, said control unit having an RF receiver, a controller and a second RF transmitter, wherein said RF receiver receives said first RF signal and provides a signal based on said first RF signal to said controller, wherein said controller determines whether said functional muscle has contracted based on said signal based on said first RF signal and causes said second RF transmitter to transmit a second RF signal if said controller determines that said functional muscle has contracted; and
a stimulating device located on or implanted within the body of said subject, said stimulating device having stimulation circuitry operatively associated with said denervated muscle, and an energy harvesting circuit including an antenna and conversion circuitry for receiving said RF energy and converting the received RF energy into DC power for powering said stimulation circuitry, wherein when said stimulating device receives said second RF signal said stimulation circuitry provides a stimulus to said denervated muscle to cause said denervated muscle to contract.
